(19) United States
(12) Patent Application Publication
(10) Pub. No.: US 2005/0100513 A1
(43) Pub. Date: May 12, 2005

(54) Homeopathic method and system for treating nicotine addiction

(76) Inventors: Mary Beth Watkins, Lummi Island, WA (US); Nicole Slater, Bellingham, WA (US)

Correspondence Address: RUBEN C. DELEON;WINSTEAD SECHROST & MINICK P.C., P.O. BOX 50784, DALLAS, TX, 75201, US

(21) Appl. No.: 10/706,769

(22) Filed: Nov. 10, 2003

(51) Int. Cl. A61K 9/68; A61K 9/20; A61K 35/78

(52) U.S. Cl. 424/48; 424/725; 424/750; 424/738; 424/464

(57) ABSTRACT

The present invention includes a method for aiding an individual in the cessation of nicotine use. The method has the steps of administering a first homeopathic composition to the individual. The first homeopathic composition is formulated to reduce nicotine craving by the individual. A second homeopathic composition is contemporaneously administered in conjunction with the first homeopathic composition. The second homeopathic composition formulated to detoxify the individual of residual nicotine and nicotine byproducts.

### TECHNICAL FIELD OF THE INVENTION

The present invention relates to anti-smoking treatments and in particular to a homeopathic method and system for treating nicotine addiction.

### BACKGROUND OF THE INVENTION

Without limiting the scope of the invention, its background is described in connection with treatments to help individuals quit smoking and is best exemplified by methods and systems to treat nicotine addiction.

The U.S. Surgeon General has determined that cigarette smoking is a major risk factor in coronary artery disease and is the cause of approximately 30% of all cancer deaths. Tobacco chewing has been shown to cause cancers of the mouth and throat. Because of the undesirable effects of tobacco smoking or tobacco chewing, many devices and drugs have been developed as aids for treatment of the tobacco and nicotine habit. For example, in a simulated smoking device, the tobacco therein is heated rather than burned, releasing nicotine vapor which is then drawn into the smoker's lungs. Thus, the smoker obtains the desired nicotine, but without also ingesting the full range and concentration of harmful products of burning tobacco. One such simulated smoking device using a source of vaporizable nicotine is disclosed in U.S. Pat. No. 4,284,089 issued to Ray.

Other simulated smoking devices contain substances which microencapsulate materials that simulate the taste and aroma of tobacco, and which are then released by squeezing or crushing the device. Such devices often do not raise the nicotine level in the blood sufficiently to satisfy the desire for nicotine, and thus are ineffective as aids to stop smoking. Other disadvantages include irritation of the mucosa, which is intolerable to some patients, and the bad taste of nicotine introduced orally. Additionally, these nicotine-based devices do not address the need to detoxify a smoker's body from the adverse affects of long-term nicotine use.

Alternatively, tobacco concentrates have been processed into tablets or gum which may be sucked or chewed in the mouth of the user, the nicotine being absorbed into the user's body through the lining of the mouth. The tobacco concentrates have a bad taste and may cause mouth ulcers and heartburn. Difficulties associated with oral administration of nicotine include nausea, rapid nicotine degradation, and irregular and unpredictable blood plasma levels. After administration of nicotine gum, effective plasma levels of nicotine may not be obtained for up to one hour. This delay may not be tolerable for an addicted smoker.

Transdermal patches have also been used as aids in the reduction of incidence of tobacco smoking or chewing. These patches contain tobacco or tobacco by-products, as described in U.S. Pat. No. 4,821,745 issued to Rosen et al, or they contain nicotine, as described in U.S. Pat. No. 4,839,174 issued to Baker et al, U.S. Pat. No. 4,908,213 issued to Govil and Kohlman, and U.S. Pat. No. 4,943,435 issued to Baker et al. Patches containing nicotine have been used in conjunction with gum containing nicotine, as described in U.S. Pat. No. 5,135,753 issued to Baker et al. One disadvantage to using a transdermal patch containing nicotine is that nicotine is a known skin irritant, and transdermal patches containing nicotine often cause itching.

In addition to the above-described drawbacks and disadvantages, all of these devices and methods suffer from a reliance on nicotine as an aid in controlling nicotine craving. Adversely, nicotine is the addictive agent that smokers are attempting to quit using. The use of nicotine as an aid in controlling nicotine addiction can cause addiction to the gum or patch itself. There is also the potential for increased addiction if the patient continues regular use of tobacco while chewing the gum or wearing the patch. Furthermore, nicotine is a known toxin with profound physiological effects on the body, including increasing blood pressure and heart rate.

The use of herbs in conjunction with transdermal patches is known in the art. A metal-based transdermal patch, applied at an acupuncture point in conjunction with a magnetic field, and containing a homeopathic mixture of at least one herb has been disclosed in U.S. Pat. No. 5,162,037 issued to Whitson-Fischman. The patch is impregnated with a homeopathic mixture of at least one herb, herbal extract or other component such as pineal gland.

The herb Plantago major, for example, has been known as a tobacco deterrent for many years. Clinical trials have also found that oral administration of Plantago major extract caused an aversion to tobacco in human subjects who were heavy smokers. It is known in the art to place the herb Plantago major in a liquid composition or in a solid form for oral ingestion to deter smoking.

Although many of the above-mentioned products address the issue of nicotine replacement or substitution, one of the difficulties of overcoming nicotine addiction is the profound physiological effects of the body when a smoker attempts to stop using tobacco products and experiences withdrawal from nicotine. Individuals suffering from nicotine withdrawal commonly experience some form of depression which is associated with withdrawal from an addictive substance. To alleviate the depressive effects of nicotine withdrawal, pharmaceutical anti-depressants, such as bupropion, are sometimes administered.

Pharmaceutical anti-depressants are often administered to smokers who are attempting to reduce their addiction to nicotine-containing products, such as cigarettes, cigars, chewing tobacco, etc. The use of pharmaceutical anti-depressants, e.g., bupropion, has a disadvantage in that the user is exposed to the side-effects which are commonly associated with such pharmaceuticals. For example, common side effects experienced during the use of bupropion are: (1) the user may experience sexual dysfunction; (2) dry mouth; (3) the user is subjected to a level of toxicity due to oral ingestion of the drug; and (4) bupropion has mutagenic effects which can be associated with birth defects.

Tobacco aversion, or a reduction in craving, may be accomplished by oral ingestion of compounds that are intended to aid in the cessation of tobacco use. As is known in the related arts, pharmaceutical compounds or other compositions may be dispersed by numerous methods. For example, pharmaceutical or other compositions may be compressed into tablets and orally ingested.

Accordingly, there is a need for a treatment for nicotine addiction that does not subject an addicted user to further or increased nicotine usage. Additionally, there is a need for a treatment for nicotine addiction that addresses detoxifying a nicotine user. There is a further need for a nicotine treatment that does not contain nicotine, tobacco byproducts or harsh chemicals that may have adverse side effects.

### SUMMARY OF THE INVENTION

A method to treat nicotine addiction according to one embodiment of the present invention includes a method for aiding an individual in the cessation of nicotine use. The method has the steps of administering a first homeopathic composition to the individual. The first homeopathic composition is formulated to reduce nicotine craving by the individual. A second homeopathic composition is contemporaneously administered in conjunction with the first homeopathic composition. The second homeopathic composition is formulated to detoxify the individual of residual nicotine and nicotine byproducts.

A homeopathic composition for treating nicotine use according to one embodiment of the present invention has a pharmaceutically effective amount of Caladium seguinum; a pharmaceutically effective amount of Daphne indica; a pharmaceutically effective amount of Plantago major; a pharmaceutically effective amount of Cinchona officinalis; a pharmaceutically effective amount of Lobelia inflata; a pharmaceutically effective amount of Nux vomica; a pharmaceutically effective amount of Staphysagria; a pharmaceutically effective amount of Calcarea Phosphorica; and a pharmaceutically effective amount of Ignatia amara.

A homeopathic composition for treating nicotine use according to one embodiment of the present invention has a pharmaceutically effective amount of Avena sativa; a pharmaceutically effective amount of Euphorbium officinarum; a pharmaceutically effective amount of Ignatia amara; a pharmaceutically effective amount of Lobelia inflata; a pharmaceutically effective amount of Nux vomica; and a pharmaceutically effective amount of Passiflora incarnate.

According to another embodiment of the present invention, a system for treating nicotine use includes a first homeopathic composition administered to reduce nicotine cravings and a second homeopathic composition contemporaneously administered with the first homeopathic composition to detoxify a nicotine user.

### BRIEF DESCRIPTION OF THE FIGURES

For a more complete understanding of the present invention, including its features and advantages, reference is now made to the detailed description of the invention taken in conjunction with the accompanying drawings in which like numerals identify like parts and in which:

FIG. 1 is a graph of results of nicotine detoxification according to one embodiment of the present invention;

FIG. 2 is a graph of results of nicotine craving relief according to one embodiment of the present invention; and

FIG. 3 is a graph of a reduction in daily nicotine craving cycle according to one embodiment of the present invention.

### DETAILED DESCRIPTION OF THE INVENTION

As used in the specification, the term “pharmaceutically effective amount” is defined as an amount that perceptively yields a particular desired result in a user. The term “cessation” is defined as quitting for a period of time. The term “detoxify” is defined as a perceptible reduction of nicotine or other smoking-related toxins in a user. The term “reduce nicotine craving” is defined as a reduction in nicotine craving as compared to nicotine craving before administering the described method or system.

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

The present invention addresses the deficiencies of the prior art by providing an innovative method and system that administer two unique homeopathic compounds to individuals who are addicted to nicotine. This two-part solution better addresses the needs and hardships of individuals who are attempting to combat their nicotine addiction. In one embodiment of the present invention a homeopathic composition used in the first part of the treatment may be delivered orally to an individual in the form of chewing gum, for example. The homeopathic composition may be contained in a coating of the gum or it may be incorporated into the gum base. Delivery of compositions or medications using gum as a carrier is known in the art. Other methods of orally delivering a composition will be apparent to those having ordinary skill in the art of medical pharmaceutical delivery systems.

The homeopathic composition of the first part of the system is formulated to reduce the cravings associated with nicotine addiction. The homeopathic composition of the present invention, unlike many prior art treatments, does not contain nicotine. The homeopathic composition of the first part of the system contains natural ingredients that individually contribute to alleviating an individual's symptoms of nicotine withdrawal.

For example, the first part of the system may be administered to the user in the form of gum. Other methods of administering the first part of the system to the user may also be used. For example, the first part of the system may be administered by a chewable lozenge, a nasal spray, a throat spray and the like. Other methods of administering the first part of the system will be apparent to those having skill in the art of pharmaceutical delivery. The gum, however, is beneficial because it occupies an addicted user with oral stimulation. In some cases, orally stimulating the user can relieve stress or anxiety that has formerly been addressed by a cigarette or tobacco in the user's mouth. As a result, simply chewing the gum may have beneficial effects to help the user stop using orally stimulating tobacco products.

The action of chewing gum alone, however, does not typically address the physiological characteristics of nicotine addiction. Consequently, the gum may have a homeopathic composition contained in the gum or the coating of the gum that helps alleviate nicotine cravings. For example, the gum may contain pharmaceutically effective amounts of Caladium seguinum, Daphne indica, Plantago major, Cinchona officinalis, Lobelia inflata, Nux vomica, Staphysagria, Calcarea Phosphorica and Ignatia amara. These ingredients may be combined in various proportions to address multiple conditions associated with nicotine detoxification and withdrawal. Caladium seguinum may help to alleviate nervous anxiety that occurs during nicotine withdrawal, for example. It may also treat respiratory conditions that have been caused by smoking. Plantago major may help treat insomnia and depression that result from nicotine withdrawal. Additionally, it may reduce irritability that may be experienced by users that are attempting to quit smoking or using tobacco products. [0025] The homeopathic composition of the second part of the system may be administered to a nicotine user to detoxify the user's body from accumulated nicotine and other residual tobacco compounds. Similar to the first part of the system, the second homeopathic composition does not contain nicotine. The second part of the system is preferably administered to the user as a chewable tablet. Other forms of administering the second homeopathic composition will be apparent to those having ordinary skill in the art of pharmaceutical delivery systems.

The homeopathic composition of the second part of the system may include pharmaceutically effective amounts of Avena sativa, Euphorbium officinarum, Ignatia amara, Lobelia inflata, Nux vomica and Passiflora incarnata. These ingredients may be combined in various proportions to address multiple conditions associated with nicotine detoxification.

Other homeopathic remedies for nicotine addiction may also be used in the first and second parts of the system. For example, Spongia, Sepia, Colchicum, Spigelia, Abies nigra, Ruta, Arsenicum Album, Staphysagria, Caladium, Taraxicum, Gelsemium, Thuja, Ignatia, Bryonia, Clematis, Colchicum, Phosphorus, Coffea cruda, Cyclamen, Euphorbium, Gelsenium, Helonias, Ipecacuanha, Magnesium carbonicum, Natrum muriaticum, Pulsatilla, Chinchona and Lobelia may be added to the first and second parts.

A more detailed presentation of the type, amount and effect of each homeopathic ingredient is presented in Tables 1A and 1B. In Table 1A, preferred homeopathic ingredients for a detoxifying chewable tablet are listed. The homeopathic ingredients may be combined in homeopathic relationships indicated after the ingredient name. For example, Avena sativa 6× may be combined with Euphorbium officinarum 6×, Ignatia amara 30×, etc. in different volumetric percentages of each homeopathic ingredient and may be added at varying ratios, such as 2:1:1 according to homeopathic pharmacy practices to achieve the desired effects of the resulting composition.

Similarly, Table 1B lists the various ingredients that may be included in an anti-craving gum. Caladium seguinum 4×, 12×, 30× may be combined with Daphne indica 4×, Plantago major 4×, etc. at varying ratios according to homeopathic pharmacy practices. Different volumetric percentages of the homeopathic ingredients may be added to the composition according to the desired effects.

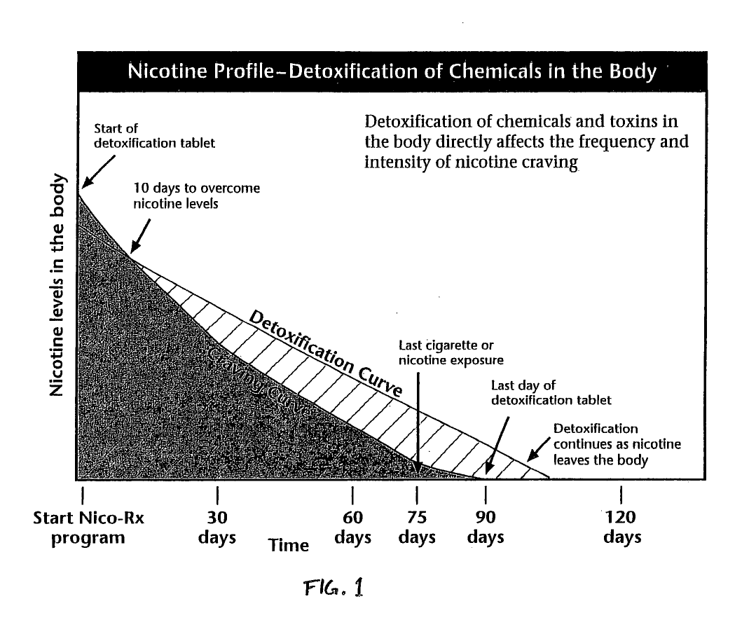

Turning now to FIG. 1, the graph depicts how the nicotine levels in a user's body are reduced over the course of using the nicotine detoxification system according to one embodiment of the present invention. For example, during the first month of using the system, a user is directed to ingest two (2) tablets, four (4) times daily. Concurrently with the tablets, the user is directed to chew one (1) piece of the gum every one (1) to two (2) hours. Within ten (10) days of beginning this system, nicotine levels in the user's body are reduced to a point at which nicotine cravings begin to subside.

During the second month of the system, the user may reduce dosages of the homeopathic compositions to one (1) piece of gum every two (2) to four (4) hours and two (2) tablets three (3) times daily. Ingestion of the tablets continues to detoxify the user and reduce irritability. Chewing the gum serves to alleviate nicotine cravings.

During the third month of using the system, a user may further decrease usage of the homeopathic compositions to one (1) piece of gum every four (4) hours, or as needed to relieve nicotine cravings. Additionally, tablet consumption may be reduced to one (1) tablet three (3) times daily. At the end of the three (3) month period of using the system, the user's body should be completely detoxified from residual nicotine.

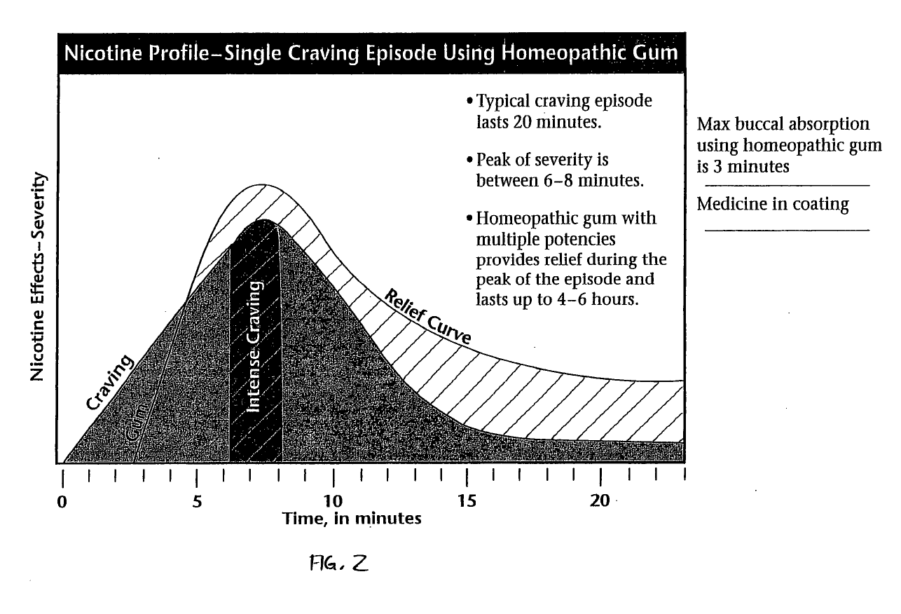

Turning now to FIG. 2, the graph depicts the intensity of a nicotine craving and how the homeopathic composition in the gum serves to alleviate the craving. When a user's craving for nicotine begins, the craving steadily increases for approximately six (6) minutes until the craving intensity peaks at approximately eight (8) minutes. If a user chews a piece of the gum within approximately three (3) minutes of the beginning of the craving, the homeopathic composition in the gum serves to satisfy the craving through the most intense period of the craving. The homeopathic composition of the gum reaches its maximum absorption in the user's body within about three (3) minutes. A typical craving lasts for about twenty (20) minutes. The effects of the homeopathic compositions in the gum serve to alleviate nicotine cravings for approximately four (4) to six (6) hours.

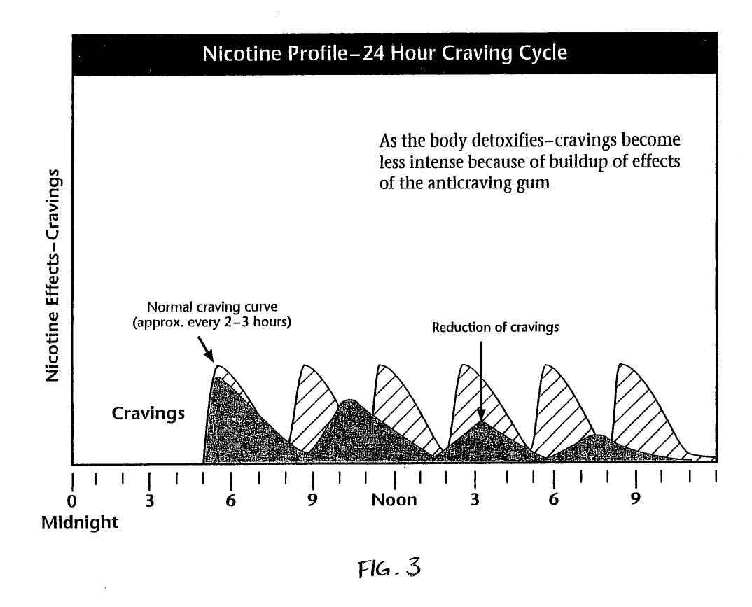

The graph depicted in FIG. 3 indicates the frequency of nicotine cravings during a typical day. As the system detoxifies a user's body, the intensity of the cravings is reduced, which aids the user in fighting the need to use nicotine.

Although this invention has been described with reference to an illustrative embodiment, this description is not intended to limit the scope of the invention. Various modifications and combinations of the illustrative embodiments as well as other embodiments of the invention will be apparent to persons skilled in the art upon reference to the description. It is therefore intended that the appended claims accomplish any such modifications or embodiments.

TABLE 1A
Nico-Rx ™

| | | | | | Tobacco | Break | Ill | In- | Nervous- | Respiratory | Short | Long |
|---|---|---|---|---|---|---|---|---|---|---|---|---|
| Tablet Ingredients | Plant | Mineral | Acute | Chronic | Addiction | Habit | Effects | somnia | ness | Support | Acting | Action |
| Avena Sativa 6× | X | | X | | X | X | X | X | X | X | | |
| Euphorbium officinarum 6× | X | | X | X | | | decongest | | | X | | X |
| Ignatia amara 12×, 30× | X | | | X | X | X | | X | X | X | X | |
| Lobelia inflata 6×, 12×, 30× | X | | X | | X | X | X | X | | X | | |
| Nux vomica 6×, 12×, 30× | X | | X | X | X | X | X | X | X | X | X | |
| Passiflora incarnata 6× | X | | X | | X | X | | X | X | X | | |

TABLE 1B

| | | | | | | Tobacco | Craving | Insomnia/ |
|---|---|---|---|---|---|---|---|---|
| Gum Ingredients | Plant | Mineral | Acute | Chronic | Tobacco | Agravates | Habit | Depression |
| Caladium seguinum | x | | x | x | x | x | aversion | |
| 4×, 12×, 30× | | | | | | | | |
| Daphne indica 4× | x | | x | slight | x | x | | |
| Plantago major 4× | x | | x | | x | | aversion | x |
| Cinchona officinalis | x | | x | x | x | x | x | |
| 6×, 12×, 30× | | | | | | | | |
| Lobelia inflata 6× | x | | x | | x | x | x | x |
| Nux vomica | x | | x | x | x | | x | x |
| 6×, 12×, 30× | | | | | | | | |
| Staphysagria 6× | x | | x | | x | x | x | |
| Calcarea Phosphorica | | x | | x | x | | x | x |
| 12× | | | | | | | | |
| Ignatia amara l 12× | x | | x | | x | x | | x |

| | Nervous | | Respi- | Short | Long | Compil- |
|---|---|---|---|---|---|---|
| Gum Ingredients | Anxiety | Irritable | ratory | Acting | Action | ments |
| Caladium seguinum | x | | x | | | |
| 4×, 12×, 30× | | | | | | |
| Daphne indica 4× | x | | x | | | |
| Plantago major 4× | | x | | | | |
| Cinchona officinalis | | | | x | | Calcarea |
| 6×, 12×, 30× | | | | | | Phosphorica |
| Lobelia inflata 6× | | | x | | | |
| Nux vomica | x | x | | x | | |
| 6×, 12×, 30× | | | | | | |
| Staphysagria 6× | | x | | | x | Calcarea |
| | | | | | | Phosphorica. |
| | | | | | | Nux vomica, |
| | | | | | | Ignatia |
| | | | | | | amara |
| Calcarea Phosphorica | x | x | x | | x | |
| 12× | | | | | | |
| Ignatia amara 12× | x | x | x | x | | Calcarea |
| | | | | | | Phosphorica |
| | | | | | | Nux vomica, |
| | | | | | | Cinchona |

1. A method for aiding an individual in the cessation of nicotine use, comprising:
administering to the individual a first homeopathic composition, the first homeopathic composition formulated to reduce nicotine craving by the individual; and
administering to the individual a second homeopathic composition, contemporaneously with administering the first homeopathic composition, the second homeopathic composition formulated to detoxify the individual of residual nicotine and nicotine byproducts.

2. The method of claim 1, wherein the first homeopathic composition is administered by a gum.

3. The method of claim 1, wherein the second homeopathic composition is administered by a chewable tablet.

4. The method of claim 1, wherein the first homeopathic composition comprises pharmaceutically effective amounts of Caladium seguinum, Daphne indica, Plantago major, Cinchona officinalis, Lobelia inflata, Nux vomica, Staphysagria, Calcarea Phosphorica and Ignatia amara.

5. The method of claim 1, wherein the second homeopathic composition comprises pharmaceutically effective amounts of Avena sativa, Euphorbium officinarum, Ignatia amara, Lobelia inflata, Nux vomica and Passiflora incarnata.

6. The method of claim 1, wherein the first homeopathic composition comprises Caladium seguinum 4×, 30×, Daphne indica 4×, Plantago major 4×, Cinchona officinalis 6×, Lobelia inflate 6×, 30×, Nux vomica 6×, Staphysagria 12×, Calcarea Phosphorica 12× and Ignatia amara 6×, 30× at homeopathically appropriate concentrations in a mixture of about equal parts.

7. The method of claim 1, wherein the second homeopathic composition comprises Avena sativa 6×, Euphorbium officinarum 6×, Ignatia amara 6×, Lobelia inflate 6×, 30×, Nux vomica 6× and Passiflora incarnate 6× at homeopathically appropriate concentrations in a mixture of about equal parts.

8. A homeopathic composition for treating nicotine use, comprising:
a pharmaceutically effective amount of Caladium seguinum;
a pharmaceutically effective amount of Daphne indica;
a pharmaceutically effective amount of Plantago major;
a pharmaceutically effective amount of Cinchona officinalis;
a pharmaceutically effective amount of Lobelia inflata;
a pharmaceutically effective amount of Nux vomica;
a pharmaceutically effective amount of Staphysagria;
a pharmaceutically effective amount of Calcarea Phosphorica; and
a pharmaceutically effective amount of Ignatia amara.

9. The homeopathic composition of claim 8, wherein the homeopathic composition is gum.

10. The homeopathic composition of claim 8, wherein the homeopathic composition comprises Caladium seguinum 4×, 30×, Daphne indica 4×, Plantago major 4×, Cinchona officinalis 6×, 30×, Lobelia inflate 6×, Nux vomica 6×, 30×, Staphysagria, Calcarea Phosphorica 12× and Ignatia amara 12× at homeopathically appropriate concentrations in a mixture of about equal parts.

11. A homeopathic composition for treating nicotine use, comprising:
a pharmaceutically effective amount of Avena sativa;
a pharmaceutically effective amount of Euphorbium officinarum;
a pharmaceutically effective amount of Ignatia amara;
a pharmaceutically effective amount of Lobelia inflata;
a pharmaceutically effective amount of Nux vomica; and
a pharmaceutically effective amount of Passiflora incarnate.

12. The homeopathic composition of claim 11, wherein the homeopathic composition is a tablet.

13. The homeopathic composition of claim 11, wherein the homeopathic composition comprises Avena sativa 6×, Euphorbium officinarum 6×, Ignatia amara 12×, 30×, Lobelia inflate 6×, 30×, Nux vomica 6×, 30× and Passiflora incarnate 6× at homeopathically appropriate concentrations in a mixture of about equal parts.

14. A system for treating nicotine use comprising:
a first homeopathic composition administered to reduce nicotine cravings in a nicotine user; and
a second homeopathic composition contemporaneously administered with the first homeopathic composition to detoxify the nicotine user.

15. The system of claim 14, wherein the first homeopathic composition is a gum.

16. The system of claim 14, wherein the second homeopathic composition is a chewable tablet.

17. The system of claim 14, wherein the first homeopathic composition comprises pharmaceutically effective amounts of Caladium seguinum, Daphne indica, Plantago major, Cinchona officinalis, Lobelia inflata, Nux vomica, Staphysagria, Calcarea Phosphorica and Ignatia amara.

18. The system of claim 14, wherein the second homeopathic composition comprises pharmaceutically effective amounts of Avena sativa, Euphorbium officinarum, Ignatia amara, Lobelia inflata, Nux vomica and Passiflora incarnata.

19. The system of claim 14, wherein the first homeopathic composition comprises Caladium seguinum 4×, 30×, Daphne indica 4×, Plantago major 4×, Cinchona officinalis 6×, 30×, Lobelia inflate 6×, Nux vomica 6×, 30×, Staphysagria 6×, Calcarea Phosphorica 12× and Ignatia amara 12× at homeopathically appropriate concentrations in a mixture of about equal parts.

20. The system of claim 14, wherein the second homeopathic composition comprises Avena sativa 6×, Euphorbium officinarum 6×, Ignatia amara 12×, 30×, Lobelia inflate 6×, 30×, Nux vomica 6×, 30× and Passiflora incarnate 6× at homeopathically appropriate concentrations in a mixture of about equal parts.
